(19) United States
(12) Patent Application Publication
(10) Pub. No.: US 2024/0320192 A1
(43) Pub. Date: Sep. 26, 2024

(54) DATA PROCESSING METHOD, DATA PROCESSING APPARATUS, AND NON-TRANSITORY COMPUTER-READABLE STORAGE MEDIUM

(71) Applicant: SHIMADZU CORPORATION, Kyoto-shi (JP)

(72) Inventors: Shiori NAGAI, Kyoto-shi (JP); Kenta ADACHI, Kyoto-shi (JP); Tomonori OSHIKAWA, Kyoto-shi (JP)

(21) Appl. No.: 18/585,661

(22) Filed: Feb. 23, 2024

(30) Foreign Application Priority Data
Mar. 20, 2023 (JP) 2023-044137

(51) Int. Cl. G06F 16/17

(52) U.S. Cl. CPC G06F 16/1734

(57) ABSTRACT

A data processing method includes a step for a computer to collect analysis file sets from a plurality of types of analyzers, the analysis file sets each including analysis data by the analyzer, and a step for the computer to tag each of a plurality of collected analysis file sets, the tag indicating a type of the analyzer, a material of a sample measured by the analyzer, preprocessing conditions of the sample, and measurement conditions of the sample.

### CROSS-REFERENCE TO RELATED APPLICATIONS

This application claims priority under 35 U.S.C. § 119 to Japanese Patent Application No. 2023-044137 filed on Mar. 20, 2023, the entire disclosure of which is incorporated herein by reference in its entirety.

### BACKGROUND OF THE INVENTION

### Field of the Invention

The present invention relates to a data processing method, a data processing apparatus, and a non-transitory computer-readable storage medium, and more specifically to a data processing method, a data processing apparatus, and a non-transitory computer-readable storage medium for processing analysis data acquired by a plurality of types of analyzers.

### Description of the Related Art

The following description sets forth the inventor's knowledge of the related art and problems therein and should not be construed as an admission of knowledge in the prior art.

International Publication No. WO 2021/235111 discloses a system for analyzing analysis data acquired by a plurality of types of analyzers cross-sectionally. In this system, the analysis data acquired by a plurality of types of analyzers are stored in a database. The data processing apparatus analyzes the analysis data stored in the database using dedicated data analysis software to generate features for use in statistical analyses or AI analyses. The data processing apparatus performs machine learning based on generated features to build a trained model.

In the above-described system, in some cases, each analyzer prepares a plurality of samples from one material, performs preprocessing on the plurality of samples under the same preprocessing conditions, and measures the samples under the same measurement conditions in order to ensure the accuracy of the analysis data. Alternatively, in some cases, one sample is measured a plurality of times under the same measurement conditions. In such cases, a plurality of analysis data in which the material of the sample, the preprocessing conditions of the sample, the type of analyzer, and the measurement conditions are all the same will be generated and stored in a database.

On the other hand, in a case where a plurality of analysis data related to a certain one material is used for machine learning, it is necessary to acquire the representative values representing the features of such a plurality of analysis data and treat the acquired representative values as the features of the material in order to reduce the load on the computer and learn efficiently.

However, since a wide variety of analysis file sets are collected from a plurality of types of analyzers and stored in the database, it is difficult to manage the analysis file sets of the same origin together.

### SUMMARY OF THE INVENTION

The present invention has been made to solve such problems, and the purpose of the present invention is to provide a data processing method, a data processing apparatus, and a non-transitory computer-readable storage medium that are capable of efficiently extracting features from a plurality of analysis data collected from a plurality of different types of analyzers.

A data processing method according to a first aspect of the present invention includes:
a step for a computer to collect analysis file sets from a plurality of types of analyzers, the analysis file sets each including analysis data by the analyzer; and
a step for the computer to assign a tag to each of a plurality of collected analysis file sets, the tag indicating a type of the analyzer, a material of a sample measured by the analyzer, preprocessing conditions of the sample, and measurement conditions of the sample.

A data processing apparatus according to a second aspect of the present invention is a data processing apparatus capable of communicating with a plurality of types of analyzers, the data processing apparatus comprises
a processor, and
a memory configured to store a program to be executed by the processor.

The processor is configured, according to the program, to
collect analysis file sets from the plurality of types of analyzers, the analysis file sets each including analysis data by the analyzer, and
assign a tag to each of the plurality of collected analysis file sets, the tag indicating a type of an analyzer, a material of a sample measured by the analyzer, preprocessing conditions of the sample, and measurement conditions of the sample.

A non-transitory computer-readable storage medium according to a third aspect of the present invention stores a program to be executed by a computer.

The program makes a computer execute
a step of collecting analysis file sets from a plurality of types of analyzers, the analysis file set each including analysis data by the analyzer, and
a step of assigning a tag to each of the plurality of collected analysis file sets, the tag indicating a type of an analyzer, a material of a sample measured by the analyzer, preprocessing conditions of the sample, and measurement conditions of the sample.

The above and other objects, features, aspects, and advantages of the present invention will become apparent from the following detailed description of the present invention understood in connection with the accompanying drawings.

### BRIEF DESCRIPTION OF THE DRAWINGS

The preferred embodiments of the present disclosure are shown by way of example, and not limitation, in the accompanying figures.

FIG. 1 is a schematic diagram describing a configuration example of an analysis system according to an embodiment of the present invention.

FIG. 2 is a schematic diagram showing a hardware configuration example of an information processing apparatus and a data processing apparatus.

FIG. 3 is a schematic diagram of a functional configuration of an information processing apparatus and a data processing apparatus.

FIG. 4 is a diagram for explaining one example of a data structure of an analysis file DB.

FIG. 5 is a diagram for explaining tagging processing by a tagging unit.

FIG. 6 is a view showing one example of a display screen of a display unit.

FIG. 7 is a diagram showing one example of a feature table.

FIG. 8 is a flowchart explaining steps performed by a data processing apparatus.

FIG. 9 is a flowchart explaining a first example of tagging processing.

FIG. 10 is a flowchart explaining a second example of tagging processing.

FIG. 11 is a schematic diagram showing one example of a UI screen displayed on a display unit.

FIG. 12 is a flowchart explaining a third example of tagging processing.

### DETAILED DESCRIPTION OF THE PREFERRED EMBODIMENTS

Hereinafter, some embodiments of the present invention will be described with reference to the attached drawings. Note that, hereinafter, the same or equivalent part in the figures is assigned by the same reference symbol, and the description thereof will not be repeated.

### <Configuration Example of Analysis System>

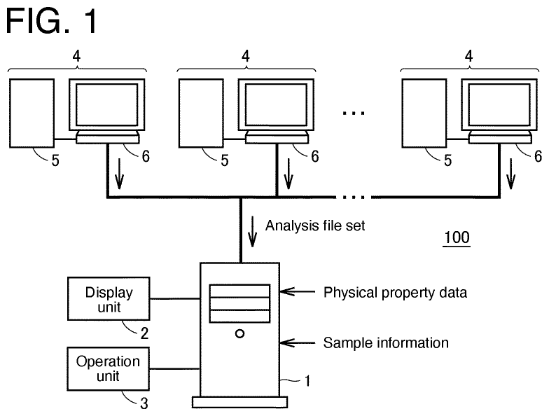

FIG. 1 is a schematic diagram for explaining a configuration example of an analysis system according to an embodiment of the present invention. The analysis system according to this embodiment can be applied to a system for analyzing analysis data acquired by a plurality of analyzers cross-sectionally. As shown in FIG. 1, this analysis system 100 is equipped with a plurality of analyzers 4 and a data processing apparatus 1.

The plurality of analyzers 4 each performs a measurement of a sample. The plurality of analyzers 4 includes a plurality of types of analyzers. In one aspect, the plurality of analyzers 4 includes a liquid chromatograph (LC), a gas chromatograph (GC), a liquid chromatograph mass spectrometer (LC-MS), a gas chromatograph mass spectrometer (GC-MS), a pyrolysis gas chromatograph mass spectrometer (Py-GC/MS), a scanning electron microscope (SEM), a transmission electron microscope (TEM), an energy dispersive X-ray fluorescence analyzer (EDX), a wavelength dispersive fluorescent X-ray analyzer (WDX), a nuclear magnetic resonator (NMR), a Fourier transform infrared spectrophotometer (FT-IR), etc. The plurality of analyzers 4 may further include a photodiode array detector (LC-PDA), a liquid chromatograph tandem mass spectrometer (LC/MS/MS), a gas chromatograph tandem mass spectrometer (GC/MS/MS), a liquid chromatograph ion trap time-of-flight mass spectrometer (LC/MS-IT-TOF), a near-infrared spectrometer, a tensile tester, a compression testing machine, an emission spectroscopic analyzer (AES), an atomic absorption analyzer (AAS/FL-AAS), a plasma mass spectrometer (ICP-MS), an organic element analyzer, a glow discharge mass spectrometer (GDMS), a particle composition analyzer, a trace total nitrogen automatic analyzer (TN), a high-sensitivity nitrogen carbon analyzer (NC), a thermal analyzer, etc. The analysis system 100 is provided with a plurality of analyzers 4, which makes it possible to perform multifaceted analyses of one sample using a plurality of different types of analysis data.

The analyzer 4 includes a device body 5 and an information processing apparatus 6. The device body 5 measures a sample as an analysis target. To the information processing apparatus 6, identification information on a sample, measurement conditions of the sample, preprocessing conditions of the sample, etc., is input.

The information processing apparatus 6 controls the measurement by the device body 5 according to the input measurement conditions. With this, analysis data based on the measurement results of the sample are acquired. The analysis data may include, for example, electron microscope images acquired by an SEM or a TEM, chromatograms and mass spectra acquired by a GC-MS or an LC-MS, as well as spectra acquired from an FT-IR or an NMR. The information processing apparatus 6 stores the acquired analysis data, together with identification information on the sample, measurement conditions, and preprocessing conditions of the sample, in a data file, and saves the data file in its built-in memory. In this specification, the data file is also referred to as “analysis file set.”

The information processing apparatus 6 is connected to the data processing apparatus 1 in a mutually communicable manner. The connection between the information processing apparatus 6 and the data processing apparatus 1 may be wired or wireless. For example, as a communication network connecting the information processing apparatus 6 and the data processing apparatus 1, the Internet can be used. With this, the information processing apparatus 6 of each analyzer 4 can transmit an analysis file set, which is a data file for each sample, to the data processing apparatus 1.

The data processing apparatus 1 is principally a device for managing the analysis data acquired by the plurality of analyzers 4. An analysis file set is input to the data processing apparatus 1 from each of the analyzers 4. It is possible to further input information on the sample (hereinafter also referred to as “sample information”) and physical property data of the sample to the data processing apparatus 1.

The sample information includes identification information to identify the sample (sample ID, sample name, etc.) and information on the sample production (hereinafter also referred to as “recipe data”). The sample recipe data may include, for example, information on the blending quantities of sample raw materials and the sample production process. The physical property data of a sample are data indicating the sample's attributes acquired by means other than the analysis by the analyzer 4.

The data processing apparatus 1 has a built-in database. The database is a storage unit for storing data exchanged between the data processing apparatus 1 and the plurality of analyzers 4, data input from outside the data processing apparatus 1, and data generated in the data processing apparatus 1. The data processing apparatus 1 stores the analysis file set, the sample information and the sample physical property data in a database for each sample, in a linked manner. Note that in the example shown in FIG. 1, it is configured such that the database is built in the data processing apparatus 1, but it can also be configured such that the database is externally attached to the data processing apparatus 1.

### <Hardware Configuration Example of Analysis System>

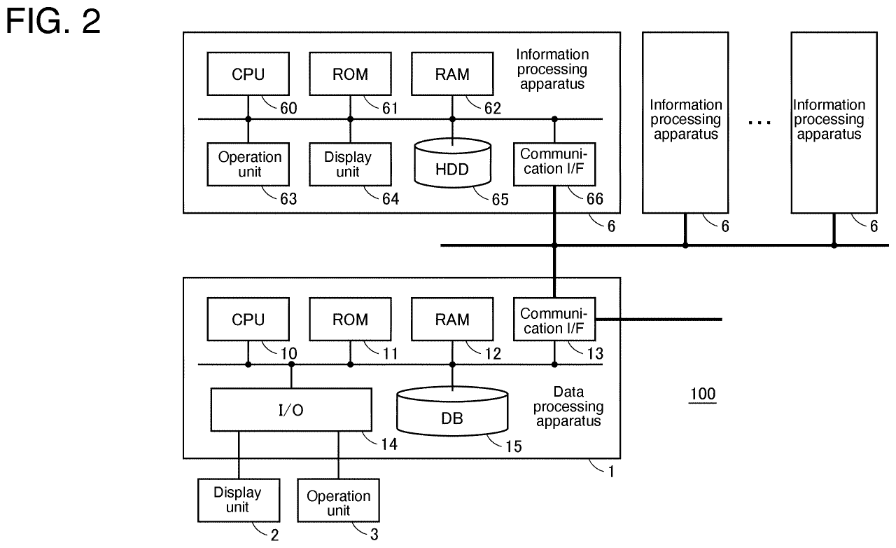

FIG. 2 is a schematic diagram showing a hardware configuration example of the information processing apparatus 6 and the data processing apparatus 1.

### (Hardware Configuration Example of Information Processing Apparatus 6)

As shown in FIG. 2, the information processing apparatus 6 is equipped with a CPU (Central Processing Unit) 60 for controlling the entire analyzer 4 and a storage unit for storing programs and data, and is configured to operate according to a program.

The storage unit includes a ROM (Read Only Memory) 61, a RAM (Random Access Memory) 62, and an HDD (Hard Disk Drive) 65. The ROM 61 stores programs to be executed by the CPU 60. The RAM 62 temporarily stores data used during the execution of a program in the CPU 60. The RAM 62 serves as a temporary data memory used as a working area. The HDD 65 is a non-volatile storage device and stores information, such as, e.g., analysis file sets, generated by the information processing apparatus 6. In addition to or instead of the HDD 65, a semiconductor memory device, such as, e.g., a flash memory, may be used.

The information processing apparatus 6 further includes a communication interface (I/F) 66, an operation unit 63, and a display unit 64. The communication I/F 66 is an interface for the information processing apparatus 6 to communicate with external devices including the device body 5 and the data processing apparatus 1.

The operation unit 63 receives an input including an instruction to the information processing apparatus 6 from the user. The operation unit 63 includes a keyboard, a mouse, and a touch panel integrally configured with the display screen of the display unit 64 to receive measurement conditions of a sample and identification information of the sample.

When setting measurement conditions, the display unit 64 can display, for example, an input screen for the measurement conditions and the sample identification information. During the measurement, the display unit 64 can display the measurement data detected by the device body 5 and the data analysis results by the information processing apparatus 6.

The processing by the analyzer 4 is realized by the respective hardware and the software executed by the CPU 60. In some cases, such software is stored in advance in the ROM 61 or the HDD 65. Further, some software may be distributed as a program product stored in a storage medium, which is not shown in the figure. The software is read out from the HDD 65 by the CPU 60 and stored in the RAM 62 in a format that can be executed by the CPU 60. The CPU 60 executes this program.

### (Hardware Configuration of Data Processing Apparatus 1)

The data processing apparatus 1 is equipped with a CPU 10 for controlling the entire apparatus and a storage unit for storing programs and data, and is configured to operate according to programs. The storage unit includes a ROM 11, a RAM 12, and a database (DB) 15.

The ROM 11 stores programs to be executed by the CPU 10. The RAM 12 temporarily stores data used during the execution of a program in the CPU 10. The RAM 12 functions as a temporary data memory used as a working area.

The DB 15 is a nonvolatile storage device, and stores data exchanged between the data processing apparatus 1 and the plurality of analyzers 4, data input from outside the data processing apparatus 1, and data generated in the data processing apparatus 1. The DB 15 is configured to include an analysis file DB 15A for storing analysis file sets collected from a plurality of analyzers 4 and a feature DB 15B for storing information on features acquired from the plurality of analysis file sets, as described below.

The data processing apparatus 1 further includes a communication I/F 13 and an input/output interface (I/O) 14. The communication I/F 13 is an interface for the data processing apparatus 1 to communicate with external devices including the information processing apparatus 6.

The I/O 14 is an interface for inputs to or outputs from the data processing apparatus 1. The I/O 14 is connected to the display unit 2 and the operation unit 3. When the processing to generate a feature table from a plurality of analysis file sets is executed in the data processing apparatus 1, the display unit 2 can display information on the processing and a user interface screen for receiving user operations.

The operation unit 3 receives inputs including user instructions. The operation unit 3 includes a keyboard and a mouse and receives sample information and sample physical property data. Note that the sample information and the sample physical property data can be received from an external device via the communication I/F 13.

### <Functional Configuration of Analysis System>

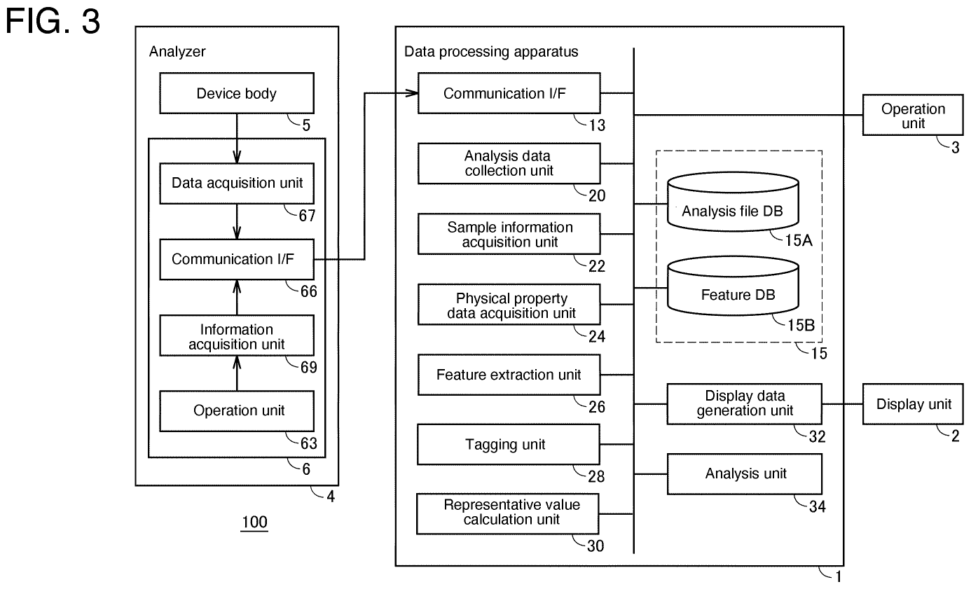

FIG. 3 is a schematic diagram showing a functional configuration of the information processing apparatus 6 and that of the data processing apparatus 1.

### (Functional Configuration of Information Processing Apparatus 6)

As shown in FIG. 3, the information processing apparatus 6 is configured to include a data acquisition unit 67 and an information acquisition unit 69. These functional configurations are realized by the CPU 60 executing a predetermined program in the information processing apparatus 6 shown in FIG. 2.

The data acquisition unit 67 acquires analysis data based on the measurement results of the sample from the device body 5. For example, in the case where the analyzer 4 is a GC-MS, the analysis data includes chromatograms and mass spectra. In the case where the analyzer 4 is an SEM or a TEM, the analysis data includes image data showing the electron microscope image of the sample. The data acquisition unit 67 transfers the acquired analysis data to the communication I/F 66.

The information acquisition unit 69 acquires the information received by the operation unit 63. Specifically, the information acquisition unit 69 acquires information indicating sample identification information, sample measurement conditions, and sample preprocessing conditions. The sample identification information includes, for example, the sample name, the product name, the model number, and the serial number of the product to be sampled. The sample measurement conditions include device parameters including the name and the model number of the analyzer to be used, and measurement parameters indicating the measurement conditions, such as, e.g., voltage and/or current application conditions and temperature conditions. Preprocessing of a sample means processing of a sample to be performed prior to performing an analysis or a measurement of the sample so that the sample becomes a state suitable for the purpose of the analysis. Preprocessing of a sample includes, for example, filtration processing and grinding processing to remove unwanted components, an ashing method to decompose and remove organic substances from a sample, and solid-phase extraction processing performed prior to a liquid chromatography analysis.

The communication I/F 66 transmits an analysis file set in which the acquired analysis data, measurement conditions, and sample identification information are combined into one file to the data processing apparatus 1.

### (Functional Configuration of Data Processing Apparatus 1)

The data processing apparatus 1 is equipped with an analysis data collection unit 20, a sample information acquisition unit 22, a physical property data acquisition unit 24, a feature extraction unit 26, a tagging unit 28, a representative value calculation unit 30, a display data generation unit 32, an analysis unit 34, an analysis file DB 15A, and a feature DB 15B. These functional configurations are realized by the CPU 10 executing a predetermined program in the data processing apparatus 1 shown in FIG. 2.

The analysis data collection unit 20 collects analysis file sets transmitted from the information processing apparatus 6 of each analyzer 4 via the communication I/F 13. The analysis file set contains the analysis data of the sample, the identification information on the sample, measurement conditions, and preprocessing conditions of the sample. The analysis data collection unit 20 stores the collected analysis file sets in the analysis file DB 15A. The analysis file DB 15A stores a wide variety of analysis file sets collected from the plurality of analyzers 4.

The sample information acquisition unit 22 acquires the sample information received by the operation unit 3. The sample information includes the sample identification information (sample ID, sample name, etc.) and the sample recipe data.

The physical property data acquisition unit 24 acquires the physical property data of the sample received by the operation unit 3. The physical property data of the sample is data indicating the sample's attributes, including, for example, a value indicating the performance of the sample and a value indicating the deterioration degree of the sample.

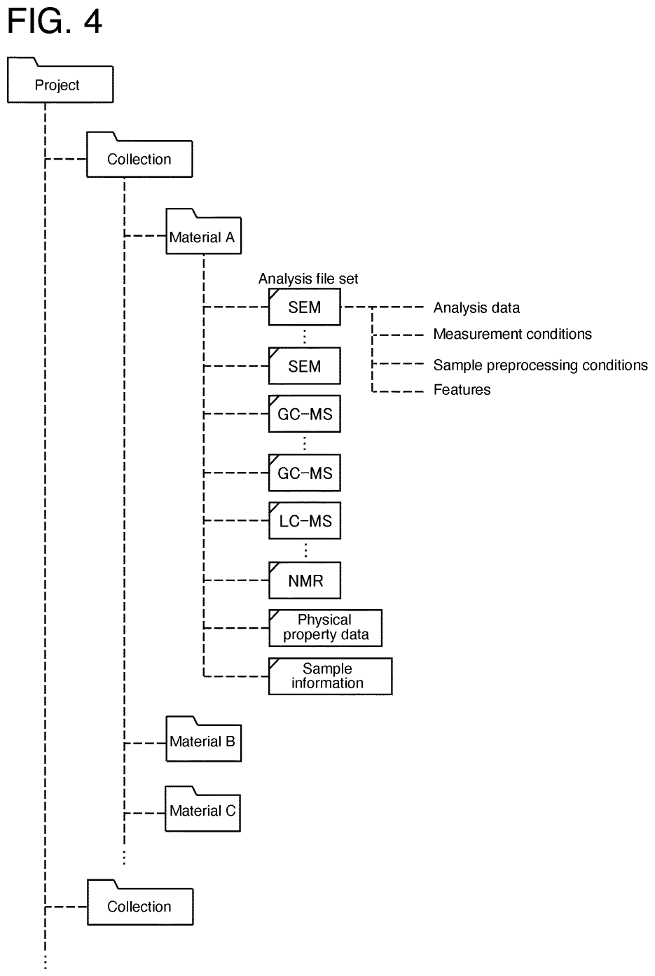

In the analysis file DB 15A, the analysis file sets collected by the analysis data collection unit 20, and the sample information and the physical property data acquired by the sample information acquisition unit 22 and the physical property data acquisition unit 24 are stored in a linked manner. FIG. 4 is a diagram for explaining one example of the data structure of the analysis file DB 15A. As shown in FIG. 4, in the analysis file DB 15A, a plurality of analysis file sets is stored in a frame called “Project.” “Project” means a frame that defines a collection of materials to be controlled to achieve a common goal, such as, e.g., a development of a new product. In this project, a plurality of analysis file sets is grouped together and stored by collection. “Collection” is a collection of materials to be used for AI analyses, statistical analyses, etc.

For example, in the case where the project is a development of a secondary battery, such as, e.g., a lithium-ion battery, the project generates a collection on positive electrode materials for a secondary battery, a collection on negative electrode materials, a collection on electrolytes, and so on. Then, a plurality of materials serving as positive electrode materials are grouped together in a collection on positive electrode materials. A plurality of materials serving as negative electrode materials are grouped together in a collection on negative electrode materials. A plurality of materials serving as electrolytes is grouped together in a collection on electrolytes.

In each collection, the plurality of analysis file sets is sorted and stored by material. Which material each analysis file set is sorted into can be determined from the sample identification information contained in the analysis file set or from the sample information associated with the analysis file set. With this, as shown in FIG. 4, a plurality of analysis file sets collected from a plurality of types of analyzers 4 is stored for one material. Sample information and physical property data are further stored for one material.

Returning to FIG. 3, the feature extraction unit 26 generates features by analyzing each analysis file set stored in the analysis file DB 15A using dedicated data analysis software. The feature data include, for example, electron microscope images acquired by an SEM or a TEM, chromatograms acquired by a GC-MS or an LC-MS, analysis data such as spectra acquired by an FT-IR or an NMR, and sample composition, concentration, molecular structure, number of molecules, molecular weight, degree of polymerization, particle diameter, particle area, number of particles, particle dispersion, peak intensity, peak area, peak slope, compound concentration, compound amount, absorbency, reflectance, transmittance, sample test intensity, Young's modulus, tensile strength, deformation amount, strain amount, fracture time, average interparticle distance, dielectric dissipation factor, elongation, spring strength, loss factor, glass dislocation temperature, and thermal expansion coefficient, calculated from the analysis data.

The feature extraction unit 26 stores the generated features in the analysis file DB 15A by associating them with the analysis file set. Note that in the example shown in FIG. 3, the feature extraction unit 26 is included in the data processing apparatus 1, but not limited thereto. The information processing apparatus 6 of each analyzer 4 may have a feature extraction unit.

The tagging unit 28 assigns a tag to the plurality of analysis file sets grouped by material. In this specification, the “tag” is used to classify and organize the plurality of file sets belonging to each material. The tag is configured to include words, symbols, or phrases that represent the origin of the analysis file set.

The “origin” of an analysis file set refers to the source of the analysis data contained in the analysis file set. The “origin” of the analysis file set includes the material of the sample to be analyzed, the preprocessing conditions of the sample, the type (instrument type) of the analyzer that measured the sample, and the measurement conditions in the analyzer. The tagging unit 28 assigns a tag representing the information to each analysis file set.

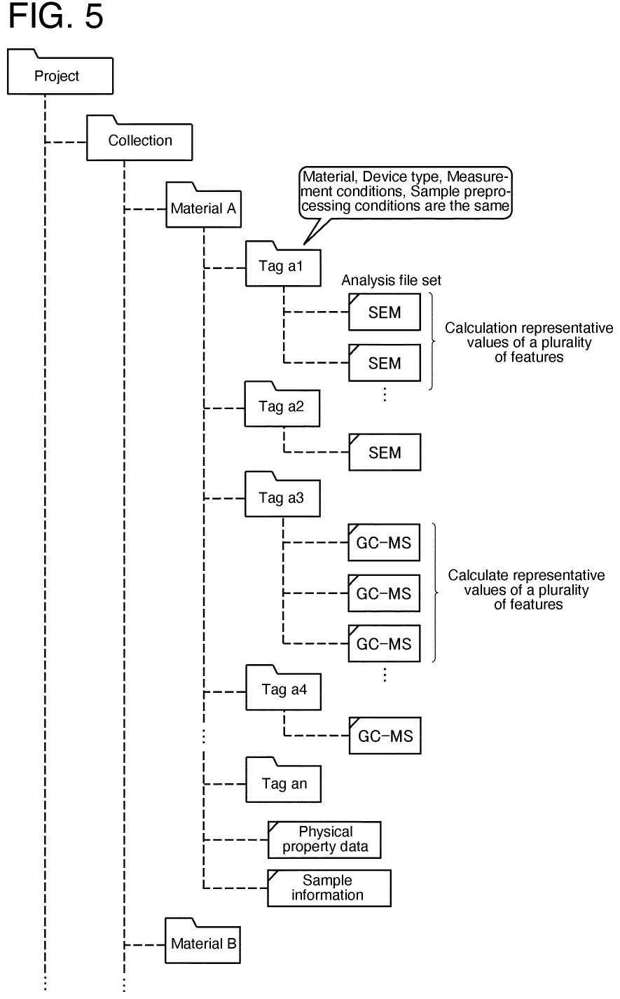

FIG. 5 is a diagram for explaining tagging processing by the tagging unit 28. As shown in FIG. 5, a tag is assigned to each of the plurality of analysis file sets belonging to one material A. As described above, the tag represents the material of the sample, the preprocessing conditions of the sample, the analyzer type, and the measurement conditions, which are the origins of the analysis file set. For example, in the case where the analysis file set contains an SEM image, the tag to be assigned to the analysis file set includes the material of the sample, the preprocessing conditions of the sample, the SEM as the instrument type, and the measurement conditions (acceleration voltage, magnification factor, etc.) of the SEM image.

Here, in each of the analyzers 4 shown in FIG. 1, in order to ensure the accuracy of analysis data, it may be sometimes configured such that a plurality of samples is prepared from one material, and the plurality of samples is preprocessed under the same preprocessing conditions and measured under the same measurement conditions. Alternatively, in some cases, for one sample, a measurement is performed a plurality of times under the same measurement conditions. For example, in the case of observing the surface structure of a sample using an SEM, a plurality of views are observed for one sample under the same measurement conditions in order to obtain the characteristics of the material as a whole. Further, in a tensile test, in some cases, a test is performed under the same measurement conditions on a plurality of test specimens made from one material, considering measurement variations due to fabrication errors of the test specimens. In these cases, a plurality of analysis data of the same origin, i.e., the material of the sample, the preprocessing conditions of the sample, the type of the analyzer, and the measurement conditions are all the same, will be generated and stored in the analysis file DB 15A.

In the data processing apparatus 1, in a case where a plurality of analysis data related to a certain one material is used for machine learning, it is necessary to acquire the representative values representing the features of such a plurality of analysis data and treat the acquired representative values as the features of the material in order to reduce the load on the computer and learn efficiently.

However, as shown in FIG. 4, a wide variety of analysis file sets are collected from a plurality of types of analyzers and stored in the analysis file DB 15A, and therefore, it is difficult to manage analysis file sets of the same origin together.

To solve these problems, in this embodiment, the tagging unit 28 is configured to assign a tag that represents its origin to each analysis file set. According to this, the same tag is assigned to a plurality of analysis file sets in which the material of the sample, the preprocessing conditions of the sample, the type of the analyzer, and the measurement conditions all match as analysis files that are identical in origin. In other words, it can be determined that the analysis file set group with the same tag is a collection of analysis file sets acquired from the perspective of ensuring the accuracy of the analysis data.

Therefore, the data processing apparatus 1 can efficiently group analysis file set groups of the same origin by using the tags assigned to each analysis file as a clue. The data processing apparatus 1 can easily calculate the representative values that serve as features from analysis file set groups of the same origin.

Returning to FIG. 3, the representative value calculation unit 30 calculates the representative values of the features extracted from each analysis file set for the analysis file set group of the same origin. The representative value is a statistic, which is a numerical value that summarizes a plurality of features. This statistic is referred to as a summary statistic. Representative values include, for example, a mean value, a median value, a mode value, a maximum value, a minimum value, a standard deviation, a variance, a skewness, a kurtosis, and so on. Among these statistic values, the mean value, the median value, the mode value, the maximum value, and the minimum value correspond to a representative value that represents the entirety of the features for each material. A standard deviation, a variance, a skewness, and a kurtosis correspond to a scatter that represents the dispersion of the features in each group.

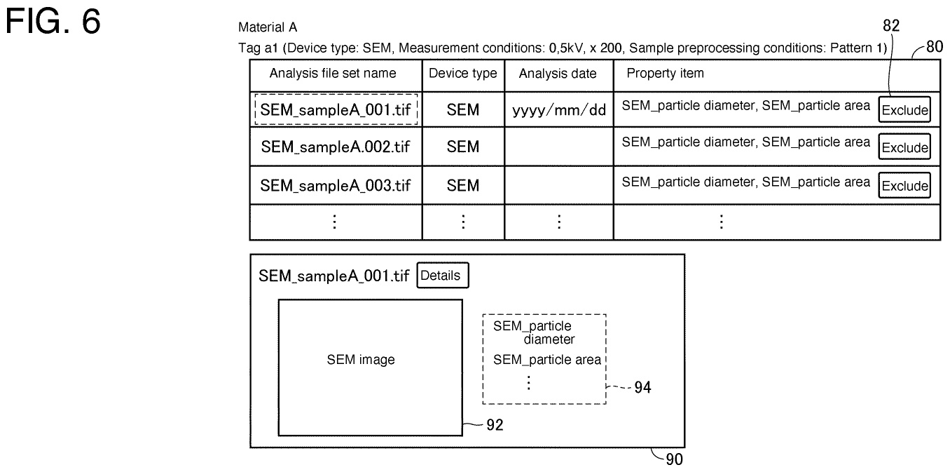

The display data generation unit 32 generates data to display the information generated by the tagging unit 28 on the display screen. Specifically, the display data generation unit 32 displays an analysis file set group with the same tag, i.e., information on analysis file set groups of the same origin, on the display unit 2. FIG. 6 is a view showing one example of a display screen of the display unit 2.

As shown in FIG. 6, the display screen of the display unit 2 displays a list showing the analysis file set group with the same tag. In the example shown in FIG. 6, a list 80 of a plurality of analysis file sets belonging to the material A, with the tag a1, is displayed.

“Tag a1” indicates that the instrument type is an SEM, the measurement conditions are an acceleration voltage of 0.5 kV and a magnification of 200×, and the sample preprocessing conditions are pattern 1. In the list 80, the analysis file set names of the analysis file sets meeting these conditions and the item names of the properties contained in each analysis file set are noted. The properties include features (SEM image, SEM_particle diameter, and SEM_particle area, etc.) extracted from an SEM image.

The user can confirm the details of the analysis file set by selecting one of the analysis file sets from the list 80 using the operation unit 3. In the example shown in FIG. 6, in response to the user's selection operation, the display screen displays a GUI (Graphical User Interface) 90 containing SEM image data 92 included in the selected analysis file set, and features 94 such as a particle diameter and a particle area calculated from the SEM image data 92.

The user can determine whether there are any measurement errors in the SEM image data 92, or whether the features 94 contain outliers, for example, by referring to the details of the displayed analysis file set. In the case where it is determined that there is a measurement error and/or an outlier, the user can exclude the analysis file set from the analysis file set group of the same origin.

Specifically, the list 80 has an icon 82 for instructing to “exclude” from the analysis file set group, for each analysis file set. In the case where it is determined that any of the SEM image data, the SEM_particle diameter, and the SEM_particle area is defective, the user can exclude the analysis file set from the analysis file set group by clicking on the icon 82 corresponding to the analysis file set containing the data. In response to this user operation, the data processing apparatus 1 excludes the analysis file set specified by the user from the analysis file set group with the tag a1.

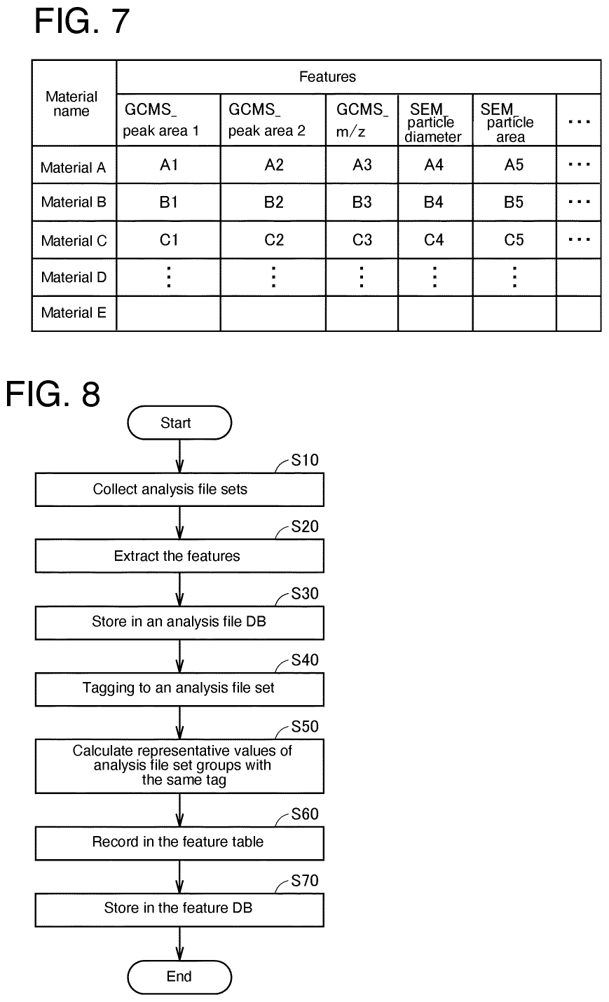

In the case where at least one analysis file set is excluded from the analysis file set group, the representative value calculation unit 30 again calculates the representative values of the plurality of features from the remaining analysis file sets in the analysis file set group. The representative value calculation unit 30 then records the calculated representative values in the feature table. In this specification, the “feature table” is a table format that summarizes the features used for analyses when performing statistical analyses and/or AI analyses of the plurality of analysis file sets. FIG. 7 is a diagram showing one example of a feature table. In the example shown in FIG. 7, the feature table records, for each material, a plurality of types of features extracted from the plurality of analysis file sets belonging to the material. The feature table can also record, in addition to the features, the physical properties of the sample and the calculated value obtained by performing arithmetic processing on at least one of the features. The generated feature table is stored in the feature DB 15B.

The analysis unit 34 performs AI analyses, statistical analyses, etc., using the feature tables stored in the feature DB 15B. The method of machine learning in the analysis unit 34 is not particularly limited. For example, known machine learning, such as, e.g., neural networks (NN: Neural Network) and support vector machines (SVM: Support Vector Machine) can be used.

### <Operation of Data Processing Apparatus 1>

Next, the processing performed by the data processing apparatus 1 will be described.

FIG. 8 is a flowchart for explaining processing steps performed by the data processing apparatus 1.

As shown in FIG. 8, initially, in Step (hereafter simply “S”) 10, the data processing apparatus 1 collects analysis file sets transmitted from the information processing apparatuses 6 of the plurality of analyzers 4 via the communication I/F 13.

In S20, the data processing apparatus 1 generates features by analyzing each of the plurality of collected analysis file sets using dedicated data analysis software.

In S30, the data processing apparatus 1 stores the plurality of collected analysis file sets in the analysis file DB 15A. In S30, the data processing apparatus 1 can store the features the sample information (sample identification information and recipe data), and the sample physical property data in a linked manner for each analysis file set. As shown in FIG. 4, a plurality of analysis file sets is sorted and stored for each material in the analysis file DB 15A.

In S40, the data processing apparatus 1 assigns a tag indicating its origin to each analysis file set stored in the analysis file DB 15A. In S40, the data processing apparatus 1 assigns a tag to each analysis file set based on the analysis data, the preprocessing conditions of the sample, and measurement conditions contained in the analysis file set. With this, as shown in FIG. 5, an analysis file set group with the same tag is generated under each material. The tagging processing procedure by the data processing apparatus 1 will be described later.

In S50, the data processing apparatus 1 calculates the representative values of the plurality of features extracted from each of the plurality of analysis file sets for the analysis file set group with the same tag. In S50, the data processing apparatus 1 can exclude at least one analysis file set from the analysis file set group according to the user's input operation to the operation unit 3, as described in FIG. 6. In the case where at least one analysis file set is excluded, the data processing apparatus 1 calculates the representative values of the plurality of features from the remaining analysis file sets.

In S60, the data processing apparatus 1 records the representative values of the plurality of calculated features in the feature table (see FIG. 7).

In S70, the data processing apparatus 1 stores the generated feature table in the feature DB 15B.

### (Tagging Processing)

Next, the processing of assigning a tag to the analysis file set (S40 in FIG. 8) will be explained with reference to a plurality of examples. In a first example, a method in which the data processing apparatus 1 automatically assigns a tag to each analysis file set will be described. In a second example, a method in which the data processing apparatus 1 assigns a tag to each analysis file set according to the user's operation will be described. In a third example, a modification of the second example will be described.

### (1) First Example

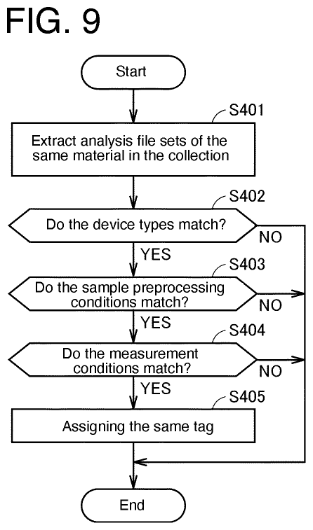

FIG. 9 is a flowchart for explaining a first example of tagging processing. In the first example, the data processing apparatus 1 automatically assigns a tag to each analysis file set based on information on the material of the sample, the preprocessing conditions, measurement conditions, and measurement data of the sample contained in the analysis file set.

Specifically, in S401, the data processing apparatus 1 extracts analysis file sets of the same material from one collection in the analysis file DB 15A. In S401, for example, a plurality of analysis file sets grouped in the material A is extracted.

Next, in S402, the data processing apparatus 1 classifies the plurality of extracted analysis file sets according to whether the device types match.

Next, the data processing apparatus 1 classifies the plurality of analysis file sets with the same apparatus type according to whether the sample preprocessing conditions match (S403) and whether the measurement conditions match (S404).

In S405, the same tag is assigned to the analysis file sets in which the type of the data processing apparatus 1, the sample preprocessing conditions, and the measurement conditions are all the same.

### (2) Second Example

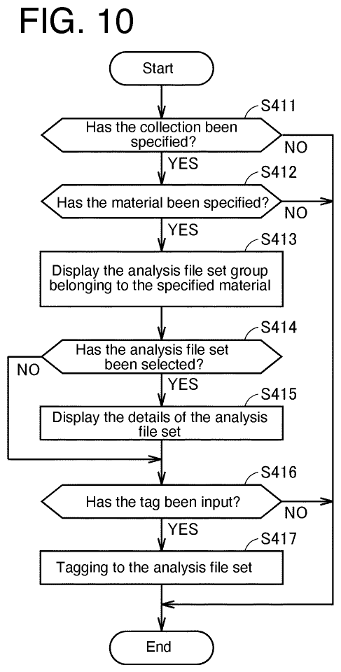

FIG. 10 is a flowchart for explaining a second example of tagging processing. In the second example, the data processing apparatus 1 is configured to provide a user interface to assist the user in tagging each analysis file set.

As shown in FIG. 10, in S411, the data processing apparatus 1 determines whether one collection has been specified for the analysis file DB 15A based on the user's input operation to the operation unit 3. Further, in S412, the data processing apparatus 1 determines whether one material has been specified. In the scene where a feature table is generated from a collection, the user can use the operation unit 3 to specify the collection and the material to be analyzed.

In the case where a collection and a material are specified (YES in S411 and S412), in S413, the data processing apparatus 1 displays the analysis file set group belonging to the specified collection and material on the display unit 2.

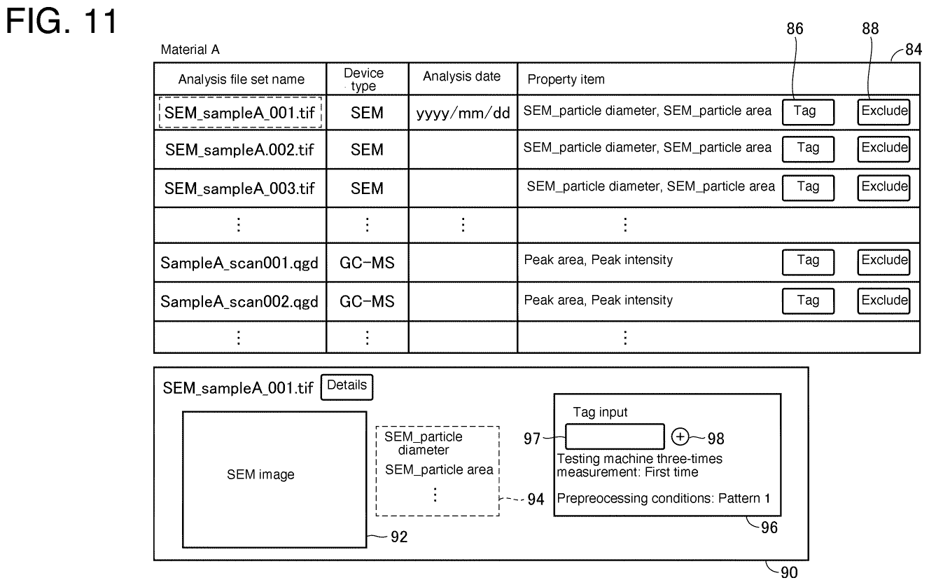

FIG. 11 is a diagram schematically showing one example of a UI screen displayed on a display unit 2. As shown in FIG. 11, the UI screen displays a list of a plurality of analysis file sets belonging to the specified material. In the example shown in FIG. 11, a list 84 of a plurality of analysis file sets belonging to a material A is displayed. The list 84 includes analysis file sets containing analysis data (SEM images) acquired by an SEM, and analysis file sets containing analysis data (chromatograms, mass spectra, etc.) acquired by a GC-MS. For each analysis file set, the name of the analysis file set and the item name of the property contained in each analysis file set are noted. Properties include features extracted from analysis data.

The user can assign a tag to each analysis file set in the list 84 using the operation unit 3. Specifically, the list 84 is provided with an icon 86 for inputting a tag for each analysis file set. When the user clicks on the icon 86 for one analysis file set, a GUI 96 for inputting a tag appears on the display screen.

The GUI 96 shows an icon 97 for inputting a tag. The user can input letters, symbols, and phrases that represent the origin of the analysis file set to the icon 97. For example, in the case where three consecutive tests were conducted on one sample under the same measurement conditions, the tag can include information indicating that the analysis data was acquired from the first of the three consecutive tests. Further, the tag can include information on the preprocessing conditions of the sample.

The GUI 96 has an icon 98 for additionally setting tag information. The user may click on the icon 98 to add additional information. The input information is displayed on the GUI 96.

Further, the user can confirm the details of the analysis file set by selecting one of the analysis file sets from the list 84 using the operation unit 3. In the example shown in FIG. 11, in response to the user's selection operation, the display screen displays a GUI 90 including SEM image data 92 included in the selected analysis file set, and features 94, such as, e.g., a particle diameter and a particle area calculated from the SEM image data 92.

The user can determine whether there are any measurement errors in the SEM image data 92, or whether the features 94 contain outliers, by referring to the details of the displayed analysis file set. In the case where it is determined that there is a measurement error and/or an outlier, the user can exclude the analysis file set from the analysis file set group. Specifically, the list 84 has an icon 88 to instruct to “exclude” from the analysis file set group, for each analysis file set. In the case where it is determined that any of the analysis data and features is defective, the user may exclude the analysis file set from the list 84 by clicking on the icon 88 corresponding to the analysis file set that contains the data. In response to this user operation, the data processing apparatus 1 excludes the analysis file set specified by the user from the plurality of analysis file sets belonging to the material A. Therefore, this analysis file set is not used when generating the feature table.

Returning to FIG. 10, in S414, the data processing apparatus 1 determines whether one analysis file set has been selected from the list 84 based on the user's operation to the operation unit 3. When one analysis file set is selected (YES in S414), in S415, the data processing apparatus 1 displays the GUI 90 containing the analysis data and the features included in the analysis file set in the GUI 90 on the display unit 2.

In S416, the data processing apparatus 1 determines whether a tag has been input based on the user's input operation to the operation unit 3. When the icon 86 in the list 84 is clicked and a tag is input in the GUI 96, in S416, it is determined to be “YES.” In response to the input of a tag (YES in S416), in S417, the data processing apparatus 1 assigns a tag to the analysis file set.

### (3) Third Example

In the second example, a configuration is described in which a tag is assigned to each analysis file set according to the user operation, but in this configuration, it may be configured such that the data processing apparatus 1 presents candidates for tags to be assigned to the analysis file set to the user.

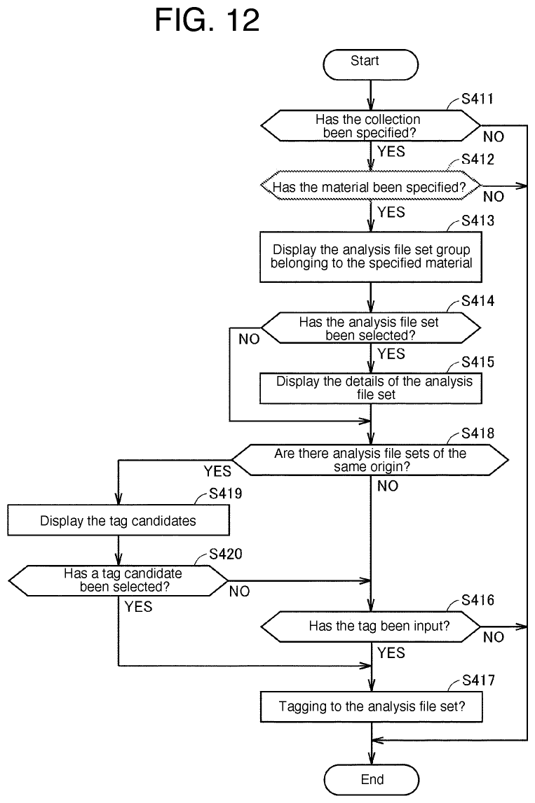

FIG. 12 is a flowchart for explaining a third example of tagging processing. The flowchart shown in FIG. 12 differs from the flowchart shown in FIG. 10 in that it includes the processing in S418 to S420.

In S418, the data processing apparatus 1 determines whether there are analysis file sets of the same origin among the plurality of analysis file sets belonging to the specified material. In S418, the data processing apparatus 1 classifies the plurality of analysis file sets based on the apparatus type, the sample preprocessing conditions, and the measurement conditions by performing the same processing as in the first example shown in FIG. 9.

In the case where there is another analysis file set that matches all of the device types, the sample preprocessing conditions, and the measurement conditions (YES in S418), the data processing apparatus 1 presents the tag assigned to another analysis file set as a candidate tag to the user in S419.

In S420, the data processing apparatus 1 determines whether the presented tag candidate is selected based on the user's input operation to the operation unit 3. In response to the selection of a candidate tag (YES in S420), the data processing apparatus 1 tags the analysis file set in S417.

In the case where there are no other analysis file sets that matches all of the device type, the sample preprocessing conditions, and the measurement conditions (NO in S418), or no tag candidate is selected (NO in S420), the data processing apparatus 1 determines, in S416, whether a tag is input based on the user's input operation to the operation unit 3. When the icon 86 in the list 84 is clicked and a tag is input in the GUI 96, in S416, it is determined to be “YES.” In response to the input of a tag (YES in S416), in S417, the data processing apparatus 1 assigns a tag to the analysis file set.

Note that in the third example, the display style of each tag may be different so that the user can distinguish between a tag assigned by the user's input operation and a tag automatically assigned by the data processing apparatus 1.

### Functions and Effects

As described above, according to the data processing method of this embodiment, in the analysis system that collects a plurality of analysis file sets from a plurality of types of analyzers and uses them for AI analyses and statistical analyses, by assigning a tag indicating its origin (sample material, sample preprocessing conditions, device type, and measurement conditions) to each analysis file set, it is possible to handle the analysis file set groups acquired as analysis file set groups of the same origin for the purpose of ensuring the accuracy of analysis data. As a result, it becomes possible to efficiently and easily calculate representative values that serve as features from the analysis file set groups of the same origin.

### Aspects

It would be understood by those skilled in the art that the exemplary embodiments described above are specific examples of the following aspects.

### (Item 1)

A data processing method according to a first aspect of the present invention includes:
a step for a computer to collect analysis file sets from a plurality of types of analyzers, the analysis file sets each including analysis data by the analyzer; and
a step for the computer to assign a tag to each of a plurality of collected analysis file sets, the tag indicating a type of the analyzer, a material of a sample measured by the analyzer, preprocessing conditions of the sample, and measurement conditions of the sample.

According to the data processing method as recited in the above-described Item 1, it is possible to efficiently manage the analysis file set group acquired to ensure the accuracy of analysis data as an analysis file set group of the same origin by using the tags assigned to each analysis file set.

### (Item 2)

In the data processing method as recited in the above-described Item 1, the analysis file set further includes features extracted from the analysis data.

The data processing method further includes:
a step for the computer to calculate representative values of a plurality of features included in an analysis file set group with the same tag; and
a step for the computer to execute machine learning using the calculated representative values.

According to the data processing method as recited in the above-described Item 2, it is possible to efficiently and easily calculate representative values that serve as features of the material from analysis file set groups of the same origin.

### (Item 3)

The data processing method as recited in the above-described Item 1 or 2, further includes:
a step for the computer to present the analysis data and the features to a user for each analysis file set in the analysis file set group.

According to the data processing method as recited in the above-described Item 3, the users can confirm the details of the analysis file set groups of the same origin.

### (Item 4)

The data processing method as recited in the above-described Item 3, further includes:
a step for the computer to receive an input operation for specifying the analysis file set to be excluded from the analysis file set group and exclude the analysis file set specified by the input operation from the analysis file set group,
wherein the step of calculating the representative values includes a step of calculating representative values of a plurality of features included in remaining analysis file sets in the analysis file set group.

According to the data processing method as recited in the above-described Item 4, it is possible to exclude analysis file sets with measurement errors or outliers from the analysis file set group to obtain representative values.

### (Item 5)

In the data processing method as recited in any one of the above-described Items 1 to 4,
the analysis file set further includes information on the type of the analyzer, the material of the sample measured by the analyzer, preprocessing conditions of the sample, and measurement conditions of the sample.

The step of assigning the tag includes a step for the computer to assign the tag to each analysis file set based on the information.

According to the data processing method as recited in the above-described Item 5, it is possible to assign a tag to each analysis file set accurately and efficiently.

### (Item 6)

In the data processing method as recited in one of the above-described Items 1 to 4, the analysis file set further includes information on the type of the analyzer, the material of the sample measured by the analyzer, preprocessing conditions of the sample, and measurement conditions of the sample.

The data processing method further comprises:
a step for the computer to present the information to a user for each of a plurality of collected analysis file sets,

The step of assigning the tag includes
a step for the computer to receive a tag input operation from the user for each analysis file set; and
a step for the computer to assign the tag received by the tag input operation to a corresponding analysis file set.

According to the data processing method as recited in the above-described Item 6, the user can tag each analysis file set while checking its origin.

### (Item 7)

In the data processing method as recited in the above-described Item 6, the step of assigning the tag includes
a step for the computer to present the tag assigned to other analysis file sets in which the type of the analyzer, the material of the sample measured by the analyzer, the preprocessing conditions of the sample, and the measurement conditions of the sample all match, as a candidate tag, to the user, based on the information contained in each analysis file set.

According to the data processing method as recited in the above-described Item 7, Users can efficiently assign tags to analysis file sets of the same origin.

### (Item 8)

A data processing apparatus according to one aspect of the present invention is a data processing apparatus capable of communicating with a plurality of types of analyzers, the data processing apparatus comprising:
a processor; and
a memory configured to store a program to be executed by the processor,

The processor is configured, according to the program, to
collect analysis file sets from the plurality of types of analyzers, the analysis file sets each including analysis data by the analyzer.

The processor assigns a tag to each of the plurality of collected analysis file sets, the tag indicating a type of an analyzer, a material of a sample measured by the analyzer, preprocessing conditions of the sample, and measurement conditions of the sample.

According to the data processing method as recited in the above-described Item 8, it is possible to efficiently manage the analysis file set group acquired to ensure the accuracy of analysis data as an analysis file set group of the same origin by using the tags assigned to each analysis file set.

### (Item 9)

A program according to one aspect of the present invention makes a computer execute:
a step of collecting analysis file sets from a plurality of types of analyzers, the analysis file set each including analysis data by the analyzer; and
a step of assigning a tag to each of the plurality of collected analysis file sets, the tag indicating a type of an analyzer, a material of a sample measured by the analyzer, preprocessing conditions of the sample, and measurement conditions of the sample.

According to the data processing method as recited in the above-described Item 9, it is possible to efficiently manage the analysis file set group acquired to ensure the accuracy of analysis data as analysis file set groups of the same origin by using the tag assigned to each analysis file set.

Further, it should be noted that in the embodiment and modifications, it is planned from the beginning of the application to combine the configurations described in the embodiments as appropriate, including combinations not mentioned in the specification, to the extent that no inconvenience or inconsistency arises.

Although some embodiments of the present invention have been described, the embodiments disclosed here should be considered in all respects illustrative and not restrictive. It should be noted that the scope of the invention is indicated by claims and is intended to include all modifications within the meaning and scope of the claims and equivalents.

1. A data processing method comprising:
a step for a computer to collect analysis file sets from a plurality of types of analyzers, the analysis file sets each including analysis data by the analyzer; and
a step for the computer to assign a tag to each of a plurality of collected analysis file sets, the tag indicating a type of the analyzer, a material of a sample measured by the analyzer, preprocessing conditions of the sample, and measurement conditions of the sample.

2. The data processing method as recited in claim 1,
wherein the analysis file set further includes features extracted from the analysis data,
wherein the data processing method further comprises:
a step for the computer to calculate representative values of a plurality of features included in an analysis file set group with the same tag; and
a step for the computer to execute machine learning using the calculated representative values.

3. The data processing method as recited in claim 2, further comprising:
a step for the computer to present the analysis data and the features to a user for each analysis file set in the analysis file set group.

4. The data processing method as recited in claim 3, further comprising:
a step for the computer to receive an input operation for specifying the analysis file set to be excluded from the analysis file set group and exclude the analysis file set specified by the input operation from the analysis file set group,
wherein the step of calculating the representative values includes a step of calculating representative values of a plurality of features included in remaining analysis file sets in the analysis file set group.

5. The data processing method as recited in claim 1,
wherein the analysis file set further includes information on the type of the analyzer, the material of the sample measured by the analyzer, preprocessing conditions of the sample, and measurement conditions of the sample, and
wherein the step of assigning the tag includes a step for the computer to assign the tag to each analysis file set based on the information.

6. The data processing method as recited in claim 1,
wherein the analysis file set further includes information on the type of then analyzer, the material of the sample measured by the analyzer, preprocessing conditions of the sample, and measurement conditions of the sample, and
wherein the data processing method further comprises:
a step for the computer to present the information to a user for each of a plurality of collected analysis file sets,
wherein the step of assigning the tag includes:
a step for the computer to receive a tag input operation from the user for each analysis file set; and
a step for the computer to assign a tag received by the tag input operation to a corresponding analysis file set.

7. The data processing method as recited in claim 6,
wherein the step of assigning the tag further includes
a step for the computer to present the tag assigned to other analysis file sets in which the type of the analyzer, the material of the sample measured by the analyzer, the preprocessing conditions of the sample, and the measurement conditions of the sample all match, as a candidate tag, to the user, based on the information included in each analysis file set.

8. A data processing apparatus capable of communicating with a plurality of types of analyzers, the data processing apparatus comprising:
a processor; and
a memory configured to store a program to be executed by the processor,
wherein the processor is configured, according to the program, to
collect analysis file sets from the plurality of types of analyzers, the analysis file sets each including analysis data by the analyzer, and
assign a tag to each of the plurality of collected analysis file sets, the tag indicating a type of an analyzer, a material of a sample measured by the analyzer, preprocessing conditions of the sample, and measurement conditions of the sample.

9. A non-transitory computer-readable storage medium storing a program,
wherein the program makes a computer execute:
a step of collecting analysis file sets from a plurality of types of analyzers, the analysis file set each including analysis data by the analyzer; and
a step of assigning a tag to each of the plurality of collected analysis file sets, the tag indicating a type of an analyzer, a material of a sample measured by the analyzer, preprocessing conditions of the sample, and measurement conditions of the sample.
